(19) United States
(12) Patent Application Publication
(10) Pub. No.: US 2011/0028840 A1
(43) Pub. Date: Feb. 3, 2011

(54) HIGH INTENSITY FOCUSED ULTRASOUND METHOD AND ASSOCIATED APPARATUS

(75) Inventor: Paul Mikus, Coto De Caza, CA (US)

Correspondence Address: REED SMITH LLP, 2500 ONE LIBERTY PLACE, 1650 MARKET STREET, PHILADELPHIA, PA, 19103, US

(73) Assignee: US HIFU, LLC, Charlotte, NC (US)

(21) Appl. No.: 12/904,800

(22) Filed: Oct. 14, 2010

Related U.S. Application Data
(63) Continuation of application No. 11/485,740, filed on Jul. 13, 2006, now abandoned.

(51) Int. Cl. A61N 7/00; A61B 8/08

(52) U.S. Cl. 600/439; 601/2

(57) ABSTRACT

A medical treatment apparatus includes a high intensity focused ultrasound transducer disposable in juxtaposition to a patient, a servomechanism operatively connected to the transducer for moving the transducer along a path, a signal generator operatively connected to the transducer for energizing same to produce focused ultrasonic pressure waves in the patient continuously during motion of the transducer along the path, and a computer operatively connected to the servomechanism for determining the path and controlling the servomechanism to move the transducer along the path. The computer is operatively connected to the generator for controlling same to energize the transducer continuously during motion of the transducer along the path.

### BACKGROUND OF INVENTION

This invention relates to high intensity focused ultrasound for use in treating patients' internal tissue structures.

The treatment of living organisms by the removal or neutralization of malignant or diseased tissue has been done over the centuries by a number of means. In the earliest days, surgical excision was done with knives or by burning with hot irons. These methods are still in use today, albeit with more sophisticated tooling.

In the early 20^{th} Century, x-ray sources were focused on internal organs which exhibited signs of malignant growths. The high radiation levels at the focal point killed the tissue in situ. X-rays were thus used both for the diagnosis and treatment of the disease. However, the radiation treatment has many undesirable side effects, such as the reduction of blood counts, the loss of hair and a general negative effect on otherwise healthy tissues.

In the last half of the 20^{th} Century, the same theory was used for treatment of disease, using acoustic energy in the form of high intensity ultrasound waves instead of x-rays. Here, ultrasound transmitters are placed either extracorporeally or on the surface of the diseased organ. The ultrasound waves converge and as they do so, the energy density, or energy per unit volume, increases. As convergence occurs, the temperature in the region of the convergence will increase due to simple thermodynamic principles. At the point of maximum energy intensity, also known as the Focal Point, the temperature of the tissue rises above the necrosis point and the tissue becomes unviable, even after the energy source is turned off.

There is a wealth of prior art on the hardware design and use of focused ultrasound waves for treatment of tissue. However, the amount tissue the transducers can treat in a single pulse of energy has limited all such designs in the marketplace.

As the waves converge, the volume of tissue at the focal point is fairly small. This actually has an advantage in that the beam can be used to treat diseased tissue close to important, healthy structures without inducing substantial collateral damage.

However, when the volume of tissue that needs to be ablated increases, the transducers must be repositioned to refocus their energy onto a different point. This learn to either having to reposition the entire transducer head or to move the transducer within a stationary outer structure so that a new volume may be treated. In either case, the total time for the procedure increases. Also, repositioning the transducer could lead to cases where gaps in the treated tissue occur, if the resulting lesions do not overlap sufficiently. When cancer is being treated, this is an unacceptable scenario.

All high-intensity focused ultrasound (HIFU) devices currently on the market include a therapy transducer, a diagnostic transducer and a computer controlled electrical signal generator with integrated diagnostic systems. In practice, both the diagnostic and therapy transducers are allowed two degrees of freedom. One degree is longitudinal with respect to the axis of the device and the second degree of freedom is radial or in an arc with respect to the axis. This radial motion is also called sector motion.

As the treatment begins, the diagnostic transducer is engaged and automatically moved to a longitudinal position that is controlled by the computer software. The physician looks at an LCD or CRT display screen and determines whether to move the view longitudinally or in sector motion. At least one diagnostic or scanning transducer is moved through this length of arc and a picture is captured and displayed on the screen.

In this diagnostic phase, the clinician will locate a target tissue region to be treated, which may be a tumor or other unwanted tissue, such as an enlarged region of a prostate gland that is closing the urethra in an otherwise healthy male. The clinician subsequently marks the area to be ablated on the screen. Pursuant to programming, the computer then moves the therapeutic transducer(s) such that the focal zone is coincident with the target tissue region. The HIFU energy is transmitted and maintained until the tissue reaches the necrosis point. At that time the HIFU energy is turned off.

In order to treat further, the probe is moved manually or, in the case of more sophisticated systems, the transducer head is moved automatically. The HIFU energy is reengaged and then turned off. This process continues until the entire volume of tissue is treated.

### OBJECTS OF THE INVENTION

It is an object of the present invention to provide an improved method and/or apparatus for delivering focused ultrasound, which is particularly useful in ablating significantly large volumes of diseased organic tissue.

Another, more particular, object of the present invention is to provide such a method and/or apparatus that substantially increases the volume of tissue treated without substantially changing the treatment method.

It is a related object of this invention to increase the volume of tissue that can be treated by a high-intensity focused ultrasound (HIFU) device in a single energy pulse.

It is a further object of this invention to provide hardware that may be used to treat tissue with a HIFU device

Another object of this invention is to provide a method of use for the hardware to treat the patient with less time and with more accuracy and completeness than previously available.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

In order to speed up HIFU treatment and to prevent gaps, the present invention contemplates that the transducer head continues to fire HIFU energy into the tissue while the transducer is moving.

In order to treat tissue, the same setup steps are done as in the standard HIFU treatment. The clinician then delineates a total volume of treatment on the CRT or LCD display. The computer turns on the system and moves the transducer head back and forth as well as radially to treat this volume. The limits of the travel both radially and longitudinally correspond to the volume painted on screen. The treatment continues until the entire volume of tissue is necrosed.

This present invention eliminates the need for moving the entire transducer physically and using the point and fire approach, which increases time of treatment.

The transducer is positioned in both radial and longitudinal coordinates by means of stepping motors or other positioning technologies that are well known to the art. Linear and/or rotary encoders may be used for positive feedback of position, speed or acceleration.

A medical treatment method with accordance with the present invention comprises (a) positioning a high intensity focused ultrasound transducer in juxtaposition to the patient, (b) energizing the transducer to focus ultrasonic pressure waves at a focal point within a target tissue region, (c) operating a computer to calculate a path of movement of the transducer, (d) further operating the computer to move the high intensity focused ultrasound transducer along the path, and (e) energizing the transducer during motion thereof along the path to destroy contiguous portions of the target tissue region.

Pursuant to another feature of the present invention, the method further comprises inputting, into the computer, information identifying the location of a target tissue region inside a patient, the computer utilizing the information to calculate the path.

It is contemplated that the method further comprises conducting a scan of internal tissues of a patient, and at least in part from the results of the scan, determining that the target tissue region should be destroyed. The conducting of the scan may includes operating the computer to transmit ultrasonic scanning waves into the patient, to analyze returning ultrasonic waves, and to project an image on a display.

The inputting of the information identifying the location of the target tissue region may be implemented by encircling an image of the target tissue region on the display.

A medical treatment apparatus comprises, in accordance with the present invention, a high intensity focused ultrasound transducer disposable in juxtaposition to a patient, a servomechanism operatively connected to the transducer for moving the transducer along a path, a signal generator operatively connected to the transducer for energizing same to produce focused ultrasonic pressure waves in the patient continuously during motion of the transducer along the path, and a computer operatively connected to the servomechanism for determining the path and controlling the servomechanism to move the transducer along the path. The computer is operatively connected to the generator for controlling same to energize the transducer continuously during motion of the transducer along the path.

The apparatus may additionally comprise at least one input peripheral operatively connected to the computer for enabling a user to input into the computer information identifying the location of a target tissue region inside a patient. The computer is programmed to determine the path in accordance with the information so that the energizing of the transducer continuously during motion thereof along the path destroys contiguous portions of the target tissue region. The input peripheral may include a touch-sensitive display screen.

It is contemplated that at least one scanning transducer is connected to the computer. The computer conducts a scan of internal tissues of a patient and is programmed to generate signals encoding images of internal tissue regions of the patient from data obtained by the scan, the display screen being operatively connected to the computer for displaying the images. The user may indicate the region of tissues to be ultrasonically excised or ablated by drawing on the screen.

### BRIEF DESCRIPTION OF THE DRAWINGS

FIG. 1 is mainly a block diagram of an ultrasonic medical treatment apparatus in accordance with the present invention, including a transducer member.

FIG. 2 is a schematic partial side elevational view of the transducer member of FIG. 1, showing a translation of the transducer member.

FIG. 3 is a schematic end elevational view of the transducer member of FIGS. 1 and 2.

FIG. 4 is a schematic partial side elevational view of the transducer member of FIGS. 1-3, showing the transducer member juxtaposed to a patient and a focusing of ultrasonic waveform energy at a point in a target tissue region inside a patient.

FIG. 5 is a schematic end elevational view of the transducer member and the patient tissues of FIG. 4, also showing the transducer member juxtaposed to the patient and the focusing of ultrasonic waveform energy at the point in the target tissue region.

FIG. 6 is a schematic partial side elevational view of the transducer member of FIGS. 1-3, depicting a movement of the transducer member along a path and a continuous projection of ultrasonic waveform energy into a target tissue region inside a patient during motion of the transducer member.

FIG. 7 is a schematic end elevational view of the transducer member of FIGS. 1-4, showing a rotational movement of the transducer member and a continuous projection of ultrasonic waveform energy into the target tissue region during motion of the transducer member.

### DETAILED DESCRIPTION

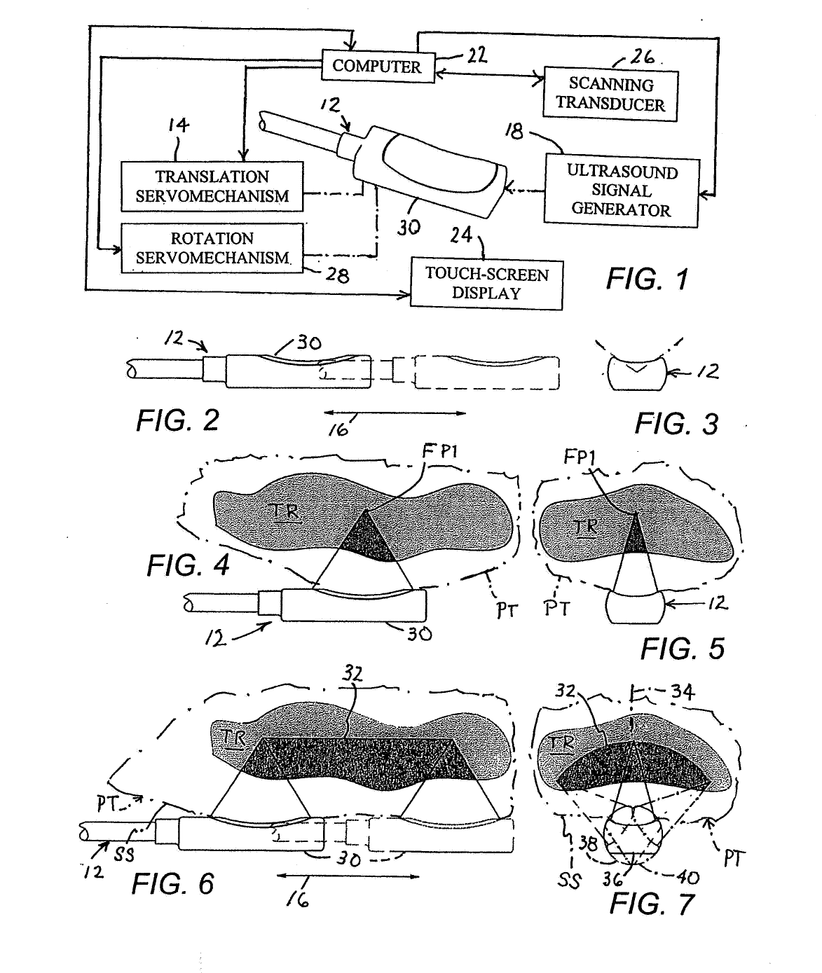

As depicted in FIG. 1, a medical treatment apparatus comprises a high intensity focused ultrasound transducer member 12 disposable in juxtaposition to a patient PT (FIG. 6). A translation servomechanism 14 is operatively connected to the transducer member 12 for moving the transducer member along a path indicated by a double headed arrow 16 (FIGS. 2 and 6). A signal generator 18 is operatively connected to transducer member 12 for energizing the same to produce focused ultrasonic pressure waves 20 (FIGS. 4 and 5) in the patient PT continuously during motion of transducer member 12 along the path 16. A computer 22 is operatively connected to servomechanism 14 for determining the path 16 and controlling the servomechanism to move the transducer along the path. Computer 22 is operatively connected to generator 18 for controlling same to energize transducer member 12 continuously during motion of the transducer member along path 16.

The medical treatment apparatus additionally comprises at least one input peripheral 24 exemplarily in the form of a touch-sensitive display screen operatively connected to computer 22 for enabling a user to input into the computer information identifying the location of a target tissue region TR inside patient PT. Computer 22 is programmed to determine path 16 in accordance with the input information so that the energizing of transducer member 22 continuously during motion thereof along path 16 destroys contiguous portions of target tissue region TR.

At least one scanning transducer 26 is connected to computer 22. Scanning transducer 26 is part of an ultrasonic scanning device that is connected to computer 22 for providing the computer with data as to internal tissue structures of the patient PT. Computer 22 conducts a scan of internal tissues of the patient PT and is programmed to generate signals encoding images of internal tissue regions (including target tissue region TR) of the patient from data obtained by the scan. Display screen 24 is operatively connected to computer 22 for displaying the images. The user may indicate the region TR of tissues to be ultrasonically excised or ablated by drawing on screen 24.

A medical treatment method utilizing the apparatus of FIG. 1 entails positioning the high intensity focused ultrasound transducer member 12 in juxtaposition to the patient PT and activating signal generator 18 to energize the transducer member to focus ultrasonic pressure waves 20 at a focal point FP1 within target tissue region TR (FIGS. 4 and 5). Computer 22 is operated to calculate path 16 of movement of the transducer member 12. Computer 22 is additionally operated to activate translation servomechanism 14 move transducer member 12 along path 16. Transducer member 12 is energized during motion thereof along path 16 to destroy contiguous portions of the target tissue region. Transducer member 12 may be energized continuously or in pulses (discrete intervals) during the motion. The energizing of transducer member 12 may commence at the time that motion along path 16 is begun or may commence at an earlier or later time.

Path 16 need not be a linear path but may trace a curve along a skin surface SS of the patient PT.

The apparatus of FIG. 1 additionally comprises a rotary servomechanism 28 operatively connected to computer 22 and to transducer member 12 for rotating or pivoting at least a head 30 of the transducer member. Thus, a tissue-destruction path 32 (FIGS. 6 and 7) inside target region TR may extend from one side to the other of a plane or contour 34 (FIG. 7) containing path 16 and oriented perpendicularly to the patient's skin surface SS. FIG. 7 shows transducer head 30 oriented in a perpendicular position 36 (in plane or contour 34) and further depicts two extreme angled orientations 38 and 40.

The inputting of the information identifying the location of target tissue region TR may be implemented by encircling an image of target tissue region TR on the display screen 24. Alternatively, a user may input information or instructions into computer 22 via peripherals other than touch-sensitive display screen 24. Such peripherals include a keyboard, a mouse, a laser pointer, etc. (none shown). The information or instructions at least in part identify the location of target tissue region TR inside patient PT. Computer 22 utilizes this information to calculate path 16.

As indicated above, computer 22 may conduct a scan of internal tissues (for instance, an ultrasonic scan) of patient PT and display an image of the patient's internal tissue structures on screen 24 or other monitor. In the case of an ultrasonic scan, computer 22 controls the transmission of ultrasonic scanning waves into patient PT, e.g., via transducer 26, analyzes returning ultrasonic waves, and projects an image on display screen 24.

In order to speed up HIFU treatment and to prevent gaps, transducer head 30 continues to fire HIFU energy into the target tissue region TR while the transducer member 12 is moving along path 16.

In order to treat tissue, the same setup steps are done as in the standard HIFU treatment. The clinician then delineates a total volume of treatment on the CRT or LCD display. Computer 22 turns on the system and moves the transducer head 30 back and forth as well as radially to treat this volume. The limits of the travel both radially and longitudinally correspond to the volume painted on screen. The treatment continues until the entire volume of tissue is necrosed.

This present invention eliminates the need for the point and fire approach, which increases time of treatment.

Servomechanisms 14 and 28 may include stepping motors or other positioning technologies that are well known to the art. Linear and/or rotary encoders (not shown) may be used for positive feedback of position, speed or acceleration.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-9. (canceled)

10. A medical treatment method comprising:
positioning a high intensity focused ultrasound transducer in juxtaposition to the patient;
energizing said transducer to focus ultrasonic pressure waves at a focal point within a target tissue region;
operating a computer to calculate a path of movement of said transducer;
further operating said computer to move said high intensity focused ultrasound transducer along said path; and
energizing said transducer to transmit a high-intensity focused ultrasound pulse of a destructive level of energy into said target tissue region while said transducer is moving along said path, thereby destroying contiguous portions of said target tissue region.

11. The method defined in claim 10, further comprising inputting, into said computer, information identifying the location of the target tissue region inside a patient, said computer utilizing said information to calculate said path.

12. The method defined in claim 11, further comprising:
conducting a scan of internal tissues of a patient;
and at least in part from the results of said scan, determining that said target tissue region should be destroyed.

13. The method defined in claim 12 wherein the conducting of said scan includes operating said computer to transmit ultrasonic scanning waves into the patient, to analyze returning ultrasonic waves, and to project an image on a display.

14. The method defined in claim 13 wherein the inputting of the information identifying the location of said target tissue region includes encircling an image of said target tissue region on said display.

15. A medical treatment apparatus comprising:
a high intensity focused ultrasound transducer disposable in juxtaposition to a patient;
a servomechanism connected to said transducer and configured to move said transducer along a path;
a signal generator connected to said transducer configured to energize same to produce focused ultrasonic pressure waves in the patient continuously during motion of said transducer along said path; and
a computer connected to said servomechanism and configured to determine said path and controlling said servomechanism to move said transducer along said path,
said computer being connected to said generator and configured to control same to energize said transducer to continuously of transmit a destructive level of ultrasonic vibratory energy into a target tissue region inside the patient while said transducer is moving along at least a portion of said path.

16. The apparatus defined in claim 15, further comprising at least one input peripheral operatively connected to said computer for enabling a user to input into said computer information identifying the location of a target tissue region inside a patient, said computer being programmed to determine said path in accordance with said information so that the energizing of said transducer continuously during motion thereof along said path destroys contiguous portions of said target tissue region.

17. The apparatus in claim 16 wherein said input peripheral includes a touch-sensitive display screen.

18. The apparatus defined in claim 17, further comprising at least one scanning transducer, said computer being operatively connected to said scanning transducer for conducting a scan of internal tissues of a patient, said computer being programmed to generate signals encoding images of internal tissue regions of the patient from data obtained by said scan, said display screen being operatively connected to said computer for displaying said images.
